(19) United States
(12) Patent Application Publication
(10) Pub. No.: US 2024/0108248 A1
(43) Pub. Date: Apr. 4, 2024

(54) APPARATUS AND METHODS FOR DETERMINING HUMAN PERFORMANCE

(71) Applicant: GMECI, LLC, Beavercreek, OH (US)

(72) Inventors: Bradford Everman, Haddonfield, NJ (US); Brian Bradke, Brookfield, VT (US)

(73) Assignee: GMECI, LLC, Beavercreek, OH (US)

(21) Appl. No.: 17/959,593

(22) Filed: Oct. 4, 2022

(51) Int. Cl. A61B 5/11; A61B 5/00; A61B 5/083; H04R 1/10

(52) U.S. Cl. CPC A61B 5/1118; A61B 5/083; A61B 5/6803; H04R 1/1041

(57) ABSTRACT

An apparatus for calculating human performance, wherein the apparatus includes at least a biological sensor configured to measure at least a biomarker pertains to a user, and a computing device configured to receive the at least a biomarker pertains to the user and determine a human performance measurement pertain to the user as a function of the at least a biomarker.

### FIELD OF THE INVENTION

The present invention generally relates to the field of biological feedback. In particular, the present invention is directed to apparatus and methods for determining human performance.

### BACKGROUND

Low performance of a user can impede the user from performing high stress and/or high stakes responsibilities. Low performance can result in unacceptable outcomes, such as loss of casualties, loss of life, and loss of assets.

### SUMMARY OF THE DISCLOSURE

In an aspect, an apparatus for determining human performance, wherein the apparatus includes at least a biological sensor configured to measure at least a biomarker pertains to a user, at least a processor communicatively connected to the at least a biological sensor, and a memory communicatively connected to the at least a processor, wherein the memory containing instructions configuring the at least processor to receive the at least a biomarker pertains to the user and determine a human performance measurement pertain to the user as a function of the at least a biomarker, classify a task pertaining to the user as a function of the human performance measurement, and certify the user to perform the task as a function of the human performance measurement.

In another aspect, a method for determining human performance, wherein the method includes measuring, using at least a biological sensor, at least a biomarker pertaining to a user, receiving, using at least a processor, the at least a biomarker pertaining to the user from the at least a biological sensor, and determining, using the processor, a human performance measurement pertaining to the user as a function of the at least a biomarker, classifying, using the processor, a task pertaining to the user as a function of the human performance measurement, and certifying, using the processor, the user to perform the task as a function of the human performance measurement.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary apparatus for determining human performance;

FIG. 2A is an exemplary embodiment of a perspective view of a headset with biomarker measurement capabilities;

FIG. 2B is an exemplary embodiment of a Front view of a headset with biomarker measurement capabilities;

FIG. 2C is an exemplary embodiment of a perspective view of a headset with biomarker measurement capabilities;

FIG. 3 is a block diagram of an exemplary machine-learning module;

FIG. 4 is a diagram of an exemplary embodiment of neural network;

FIG. 5 is a diagram of an exemplary embodiment of a node of a neural network;

FIG. 6 is a flow diagram of an exemplary method of determining human performance; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus for determining human performance, wherein the apparatus includes at least a biological sensor configured to measure at least a biomarker pertaining to a user, at least a processor communicatively connected to the at least a biological sensor, and a memory communicatively connected to the at least a processor, wherein the memory containing instructions configuring the at least processor to receive the at least a biomarker pertaining to the user and determine a human performance measurement pertaining to the user as a function of the at least a biomarker, classify a task pertaining to the user as a function of the human performance measurement, and certify the user to perform the task as a function of the human performance measurement.

At another high level, aspects of the present disclosure are directed to method for determining human performance, wherein the method includes measuring, using at least a biological sensor, at least a biomarker pertaining to a user, receiving, using at least a processor, the at least a biomarker pertaining to the user from the at least a biological sensor, and determining, using the processor, a human performance measurement pertaining to the user as a function of the at least a biomarker, classifying, using the processor, a task pertaining to the user as a function of the human performance measurement, and certifying, using the processor, the user to perform the task as a function of the human performance measurement.

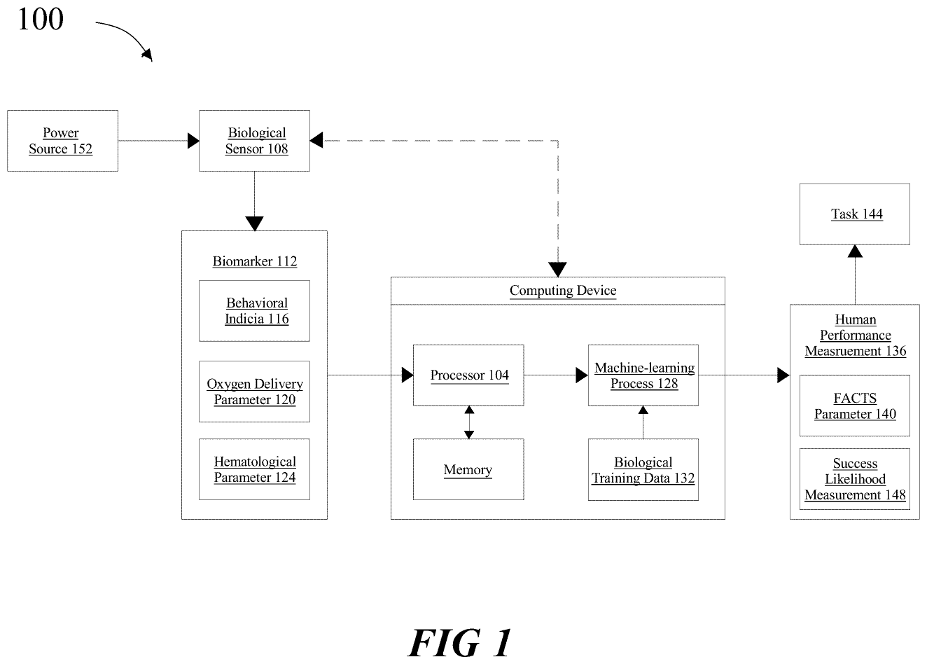

Referring now to FIG. 1, an exemplary embodiment of an apparatus 100 for determining human performance is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 may include a biological sensor 108 configured to measure at least a biomarker 112 pertaining to a user. As used in this disclosure, a “biological sensor” is a sensor device that produces an electrical output signal for purpose of sensing and monitoring biological events or changes in its environment. In some embodiments, biological sensor 108 may include one or more processors that perform one or more processing steps as described in this disclosure. In some cases, biological sensor 108 may include, without limitation, a temperature sensor, EMG sensor, ECG sensor, airflow sensor, glucometer sensor, pressure sensor, acoustic sensor, image sensor, magnetic field sensor, and the like thereof. In some embodiments, without limitation, biological sensor 108 may include a physical sensor, wherein the physical sensor is a device that measures a physical quantity. In some cases, biological sensor 108 may convert physical quantity into output signal which can be read by processor 104. In some embodiments, without limitation, biological sensor 108 may include a chemical sensor, wherein the chemical sensor is a device that converts a property of a particular analyte into a measurable signal that is proportional to the analyte concentration. In some cases, chemical sensor may recognize analyte molecule in a selective way by transforming it into an analytical electrical signal. In some cases, analyte concentration may include, without limitation, PH value, Ca+ concentration, glucose concentration of body liquid and the like thereof. In some embodiments, without limitation, biological sensor 108 may include a biosensor, wherein the biosensor is a device that combine a biological material with a suitable platform for a detection of pathogenic organisms, carcinogenic, mutagenic, toxic chemicals or for reporting a biological effect. In some cases, biosensor may include, without limitation, electrochemical biosensor, physical biosensor, optical biosensor, wearable biosensor, and the like thereof. In some embodiments, without limitation, biological sensor 108 may be used to monitor a plurality of biological parameters over time such as without limitation, biomarker 112.

With continued reference to FIG. 1, as used in this disclosure, a “biomarker” is a sign of a normal or abnormal process or a condition of the user. In some embodiments, the at least a biomarker 112 may include a behavioral indicia 116. As used in this disclosure, “behavioral indicia” is a parameter representing visible, audible, or otherwise noticeable indication of a behavior of the user. In some cases, behavior may include conscious action of the user. In other cases, behavior may include unconscious action of the user. In some embodiments, behavior may include large body moments, such as without limitation, walking, running, skipping, jumping, throwing, climbing, biking, balancing, dancing, and the like thereof. In other embodiments, behavior may include small body moments, such as without limitation, speaking, watching, typing, and the like thereof. In a non-limiting example, behavioral indicia may include an eye parameter for an eye tracking movement, wherein the eye tracking movement may include, without limitation, blinking status, blinking rate, and the like thereof. In another non-limiting example, behavioral indicia may a parameter indicate a speak pattern for an audio speak behavior of the user.

With continued reference to FIG. 1, in some embodiments, biological sensor 108 may include an eye sensor configured to detect behavioral indicia such as, without limitation, one or more eye parameters. As used in this disclosure, an “eye parameter” is a measurement of information associated with an eye. Exemplary non-limiting eye parameters may include blink rate, eye-tracking parameters, pupil location, gaze directions, pupil dilation, and the like. In some cases, eye sensor may further include an optical sensor, electromyography sensor, and the like thereof. In other embodiments, biological sensor 108 may include a speech sensor, wherein the speech sensor is configured to detect behavioral indicia such as, without limitation, one or more speech parameters. As used in this disclosure, a “speech parameter” is an element of information associated with speech. An exemplary non-limiting speech parameter is a representation of at least a portion of audible speech, for instance a digital representation of audible speech. In some cases, behavior indicia such as speech parameter may not be directly related to speech and may be partially related to breathing. In a non-limiting example, breathing sounds may be detected by speech sensor and used as behavior indicia. Eye sensor and/or speech sensor described here may be consistent with any eye sensor and/or speech sensor disclosed in U.S. patent application Ser. No. 17/731,935, filed on Apr. 28, 2022, entitled “SYSTEM AND METHODS FOR DETERMINING ACTOR STATUS ACCORDING TO BEHAVIORAL PHENOMENA”.

With continued reference to FIG. 1, in some embodiments, the at least a biomarker 112 may include an oxygen delivery parameter 120. As used in this disclosure, an “oxygen delivery parameter” is a cardiovascular and/or respiratory parameter usable to measure effectiveness of the respiratory and/or cardiovascular system in getting oxygen to user's brain. In some cases, oxygen delivery parameter 120 may include, without limitation, pulse rate, spO₂ (i.e., blood oxygen level), exhaled CO₂ level, respiration rate, any other oxygenation parameters and the like thereof. In other cases, at least a biomarker 112 may include a plurality of oxygen delivery parameters. In some embodiments, biological sensor 108 may include a human performance oxygen sensor. As used in this disclosure, a “human performance oxygen sensor” is a near-infrared spectroscopy sensor which is capable of measuring oxygenation signals. In a non-limiting example, near-infrared spectroscopy sensor may emit near-infrared light (i.e., red light) into soft tissue and measure an amount of absorbed red light and an amount of reflected red light. In some cases, oxygenation signals may include signal from, without limitation, pulse oximetry, pulse, temperature, and the like thereof. In an embodiment, human performance oxygen sensor may measure at least a biomarker 112 such as, without limitation, oxygen delivery parameter 120 or any other oxygenation parameters by positioned over an ear of the user. In another embodiment, human performance oxygen sensor may measure at least a biomarker 112 such as, without limitation, oxygen delivery parameter 120 or any other oxygenation parameters by rest behind ear on the neck of the user, over a sternocleidomastoid muscle. For instance, and without limitation, human oxygen sensor may be consistent with any human oxygen sensor disclosed in U.S. patent application Ser. No. 10/667,731, filed on Apr. 20, 2017, entitled “HUMAN PERFORMANCE OXYGEN SENSOR.”

With continued reference to FIG. 1, in some embodiments, biological sensor 108 may further include a physiological sensor, wherein the physiological senor is configured to detect an emergent physiological state based on user inspiration and expiration. As used in this disclosure, an “inspiration” is when fluid, such as, without limitation, air is taken into user respiration system. As used in this disclosure, “expiration” is a process of moving fluid, such as, without limitation, air out of user respiration system. As used in this disclosure, an “emergent physiological state” refers to any human state or condition that arises which acutely diminishes human performance of one affected. Non-limiting examples of emergent physiological states may include hypoxia, hypocapnia, hypercapnia, hyperventilation, hyperventilation, atelectasis, and the like. In a non-limiting example, physiological sensor may sense a fluid volume such as blood volume within a fluid channel, wherein the fluid channel may be a pathway for a flow of fluid within apparatus 100. Physiological sensor may be configured to send a datum or signal representing atelectasis when detected fluid volume exceed a fluid volume limit. In some embodiments, without limitation, physiological sensor may be configured to detect one or more quantities and/or percentages of gases. In some embodiments, without limitation, physiological sensor may detect a carbon dioxide level, wherein the carbon dioxide level may further be outputted to processor 104 as an electrical signal or any data type described in this disclosure. In other embodiments, physiological sensor may detect one or more gases, droplets, particulate elements, or the like, which may be indicative of health and/or physiological status of the user. In some embodiments, without limitation, physiological sensor may be connected to processor 104 and may be able to detect one or more concentrations of compounds exhaled by a user equip physiological sensor, such as, without limitation, concentrations of exhaled CO₂, exhaled volatile organic compounds and/or tVOC, or the like. In other embodiments, physiological sensor may be able to detect one or more concentrations of compounds in environment surrounding the user, such as, without limitation, CO₂ levels, VOC levels, tVOC levels, or the like. Processor 104 may determine an oxygen delivery parameter 120 as a function of one or more concentrations described above. For instance, and without limitation, physiological sensor may include an exhalation sensor, wherein the exhalation sensor may be consistent with the sensor disclosed in U.S. patent application Ser. No. 16/933,680, filed on Jul. 20, 2020, entitled “COMBINED EXHALED AIR AND ENVIRONMENTAL GAS SENSOR APPARATUS”. For another example, and without limitation, physiological sensor may include an inhalation sensor, wherein the inhalation sensor may be consistent any inhalation sensor disclosed in U.S. patent application Ser. No. 17/333,169, filed on May 28, 2021, entitled “SYSTEM AND METHODS FOR INSPIRATE SENSING TO DETERMINE A PROBABILITY OF AN EMERGENT PHYSIOLOGICAL STATE”. In a non-limiting example, a biological sensor 108 may be placed at an exhaust port of a field breathing of a user, wherein the biological sensor may include a physiological sensor. Physiological sensor may be configured to measure one or more oxygen delivery parameters 120 as a function of a carbon dioxide level, wherein the carbon dioxide level may be detected by physiological sensor when the user exhales. In general, high carbon dioxide level may lead to a low oxygen delivery parameter, and low carbon dioxide level may lead to a high oxygen delivery parameter.

With continued reference to FIG. 1, in some embodiments, the at least a biomarker 112 may include at least a hematological parameter 124. As used in this disclosure, a “hematological parameter” is a quantitative measurement related to the blood of the user. In some cases, hematological parameter may be detected by cutaneous sensor described below in this disclosure. In some embodiments, hematological parameter 124 may include measurements relate to, without limitation, blood cells, hemoglobin, blood proteins, bone marrow, platelets, blood vessels, spleen, and the like thereof. In some other embodiments, without limitation, hematological parameter 124 may describe the state of blood vessels such as arteries, veins, or capillaries, any datum describing the rate, volume, pressure, pulse rate, or other state of flow of blood or other fluid through such blood vessels, chemical state of such blood or other fluid, or any other parameter relative to health or current physiological state of user as it pertaining to the cardiovascular system. As a non-limiting example, at least a hematological parameter 124 may include a blood oxygenation level of user's blood. At least a hematological parameter 124 may include a pulse rate. At least a hematological parameter 124 may include a blood pressure level. At least a hematological parameter may include heart rate variability and rhythm. At least a hematological parameter 124 may include a plethysmograph describing user blood-flow; in an embodiment, plethysmograph may describe a reflectance of red or near-infrared light from blood. One circulatory parameter may be used to determine, detect, or generate another circulatory parameter; for instance, a plethysmograph may be used to determine pulse oxygen level (for instance by detecting plethysmograph amplitude), pulse rate (for instance by detecting plethysmograph frequency), heart rate variability and rhythm (for instance by tracking pulse rate and other factors over time), and blood pressure, among other things.

With continued reference to FIG. 1, in some embodiments, biological sensor 108 may include a cutaneous sensor, wherein the cutaneous sensor is configured to detect a cutaneous parameter as a function of a cutaneous phenomenon, wherein the cutaneous parameter is a representation of the cutaneous phenomenon. Exemplary cutaneous phenomenon may include, without limitation, skin temperature, electrical conductivity of skin, skin moisture, galvanic skin response and the like. In some cases, cutaneous sensor may further include galvanic skin sensor, skin temperature sensor, and the like thereof. For instance, and without limitation, cutaneous sensor may be consistent with any cutaneous sensor disclosed in U.S. patent application Ser. No. 17/892,542, filed on Aug. 22, 2022, entitled “SYSTEM AND METHODS FOR CORRELATING CUTANEOUS ACTIVITY WITH HUMAN PERFORMANCE”. In a non-limiting example, a biological sensor 108 may include a cutaneous sensor, wherein the cutaneous sensor may measure a hematological parameter 124, and wherein the hematological parameter 124 may include a measurement reflect at least a branch of a carotid artery. At least a hematological parameter 124 may be captured by placing biological sensor 108 in a proximity to at least a branch of carotid artery.

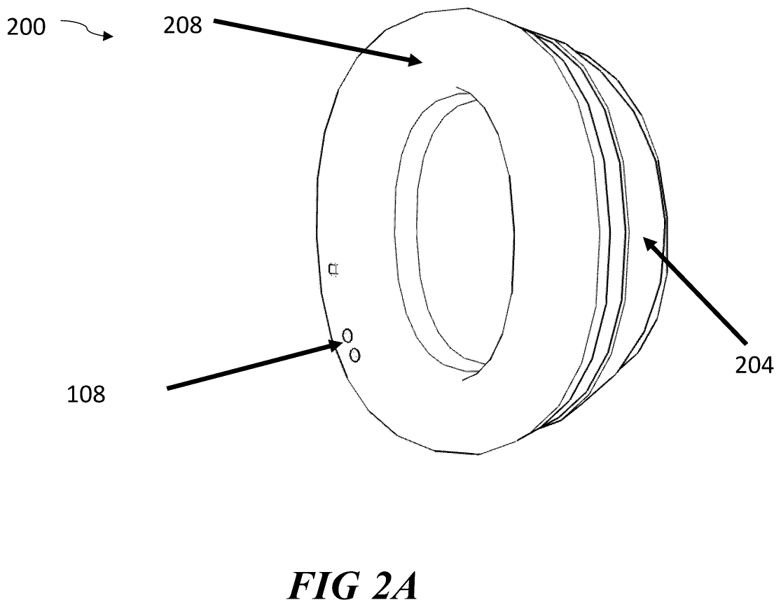

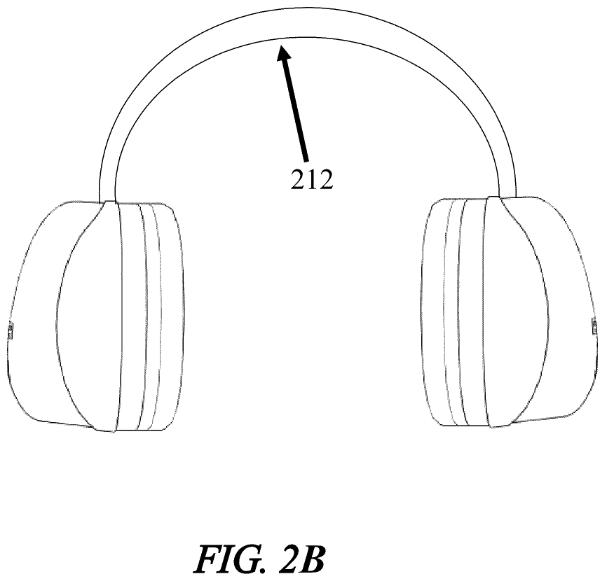

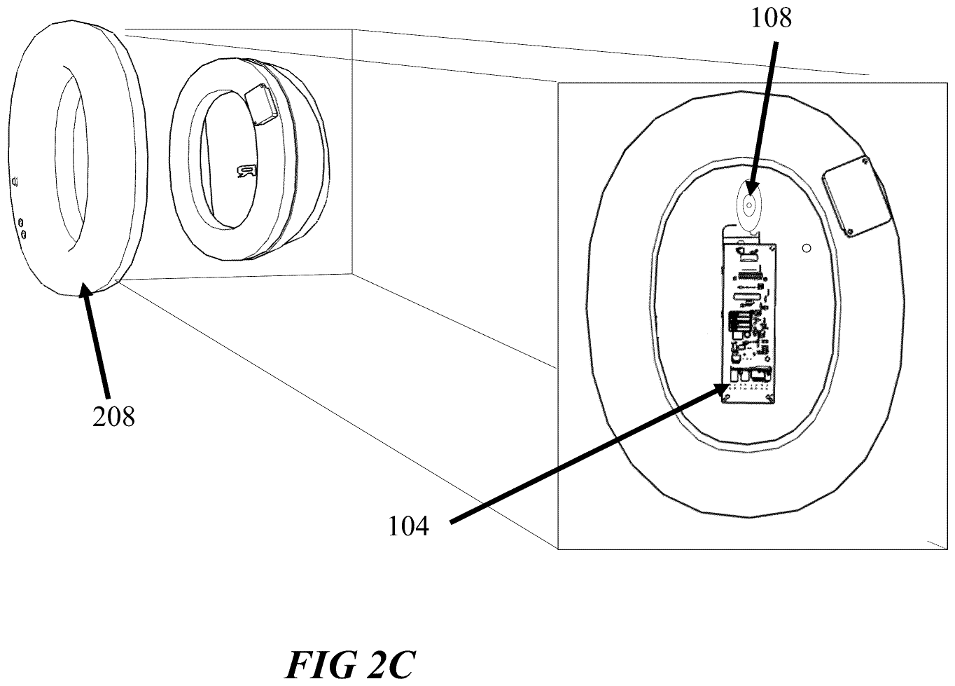

With continued reference to FIG. 1, in some embodiments, apparatus 100 may be incorporated into a standalone headset. Headset may include a plurality of housings. As used in the current disclosure, a “housing” is a rigid casing that encloses equipment. Housing may be constructed of any material or combination of materials, including without limitation metals, polymer materials such as plastics, wood, fiberglass, carbon fiber, or the like. In an embodiment, housing is shaped to conform to a particular portion of user anatomy when placed on exterior body surface. When placed to so conform, housing may position at least a sensor and/or user signaling device in a locus chosen as described in further detail below. For example, a biological sensor 108 may be mounted within housing, such that the biological sensor 108 is placed in contact with a user's skin during use. For instance, where housing is incorporated in a helmet, mask, earcup or headset, housing may be positioned at a particular portion of user's head when helmet, mask, earcup or headset is worn, which may in turn position at least a sensor and/or user signaling device at a particular locus on user's head or neck. Headset and housing are discussed in further with respect to FIG. 2. Additionally, or alternatively, biological sensor 108 may be disposed on a location on user's body for measuring biomarkers such as, without limitation, behavior indicia, oxygen delivery parameter, hematological parameter, and the like thereof. In a non-limiting example, biological sensor 108 may be a headset and positioned on user's head. In some embodiments, without limitation, biological sensor 108 may be disposed on a plurality of location on user's body. In a non-limiting example, biological sensor 108 may be a mask which includes a physiological sensor and a cutaneous sensor, wherein the physiological sensor may be placed on a field of breathing of a user and the cutaneous sensor may be placed on a forehead of the user. In some embodiments, without limitation, biological sensor 108 may measure a plurality of biomarkers such as, without limitation, behavior indicia, oxygen delivery parameter, hematological parameter, and the like simultaneously at different locations on user's body.

Still referring to FIG. 1, in some embodiments, apparatus 100 may additionally include at least a user interface. User interface may include any user interface described in this disclosure, such as without limitation a headset. User interface may be in communication with processor 104. User interface may be configured to communicate an alert to a user, for example as a function of the biomarker 112. In some cases, user interface may be configured to communicate an alert, for instance to flight crew member. In some cases, user interface 136 may be configured to communicate alert as a function of likelihood of atelectasis 120. As used in this disclosure, an “alert” is a communication to a flight crew member. Alert may alternatively be referred to in this disclosure as being or relating to a physiological alarm condition. In some cases, an alert may indicate a warning pertaining to a flight crew member's risk of atelectasis. An alert may be communicated audibly, visually, and/or haptically. In some cases, alert may include a message. As used in this disclosure, a “message” is a communication configured to communicate information. For example, in some cases, a message may communicate a procedure which a user should engage in. Alternatively, or additionally, a message may communicate a warning to a user about diminished human performance. A message may be communicated visually, audibly, and/or haptically. As used in this disclosure, a “user interface” is a system that is designed and/or configured to facilitate communication between at least a system, such as without limitation a processor 104, and a user by way of at least an output communicated to the user and/or at least an input communicated from the user. Exemplary non-limiting user interfaces include displays, audio systems, haptic systems, head mounted displays, mice, joysticks, keyboards, and the like. User interface may be configured to alert flight crew member as a function of likelihood of atelectasis. In some cases, user interface may include headphones, for example over ear headphones including an earcup. In some cases, user interface may include a bone conducting transducer, for example located within an earcup of a headphone. A “bone-conducting transducer,” as used in this disclosure, is a device or component that converts an electric signal to a vibrational signal that travels through bone in contact with the device or component to an inner ear of user, which interprets the vibration as an audible signal. Bone-conducting transducer may include, for instance, a piezoelectric element, which may be similar to the piezoelectric element found in speakers or headphones, which converts an electric signal into vibrations. In an embodiment, bone-conducting transducer may be mounted to housing in a position placing it in contact with a user's bone; for instance, where housing includes or is incorporated in an ear cup, housing may place bone-conducting transducer in contact with user's skull just behind the ear, over the sternocleidomastoid muscle. Likewise, where housing includes a headset, mask, or helmet, housing may place bone-conducting transducer in contact with a portion of user's skull that is adjacent to or covered by headset, mask, or helmet. Additional disclosure related to headphones and bone conducting transducers may be found in U.S. patent application Ser. No. 16/859,483, filed on Apr. 27, 2020, and entitled “HUMAN PERFORMANCE OXYGEN SENSOR,” the entirety of which is incorporated herein by reference.

With continued reference to FIG. 1, apparatus 100 may further include a power source 152. As used in this disclosure, a “power source” is a source of power such as, without limitation, electric power. In some cases, power source 116 may be connected to a plurality of electronic device or components such as, without limitation, biological sensor 108, processor 104, any other device requires electric power described in this disclosure, and the like thereof. In some embodiments, without limitation, power source 152 may be configured to generate an electric power. In some cases, power source 152 may include a generator, wherein the generator is a device that converts motive power (i.e., mechanical energy) into electric power. In some embodiments, power source 152 may be further configured to transmit electric power to device and/or components which requires electricity to operate, such as, without limitation, biological sensor 108, processor 104, other device requires electric power described in this disclosure, and the like thereof. In some cases, transmitting electric power may include using one or more continuous conductor. As used in this disclosure, a “continuous conductor” is an electrical conductor, without any interruption, made from electrically conducting material that is capable of carrying electrical current. Electrically conductive material may comprise copper for example. Electrically conductive material may include any material that is conductive to electrical current and may include, as a nonlimiting example, various metals such as copper, steel, or aluminum, carbon conducting materials, or any other suitable conductive material. In a non-limiting example, power source 152 may transmit electric power through a conductive wire to biological sensor and/or processor 104. Additionally, or alternatively, power source 152 may be integrated and/or embedded within biological sensor 108, and/or processor 104. In a non-limiting example, biological sensor 108 and/or processor 104 may be supplied by separate power source. In other embodiments, biological sensor 108 and/or processor 104 may share a common power source 152. In a non-limiting example, a power source 152 may be remote to biological sensor and/or processor 104 and transmit electric power through one or more continuous conductor to biological sensor 108 and/or processor 104 over a distance.

With continued reference to FIG. 1, in some embodiments, power source 152 may include one or more battery packs. A “battery pack,” as used in this disclosure, is a set of a plurality of battery cells. In some cases, battery pack may be configured in series, parallel, or a mixture of both to deliver certain voltage, capacity, and/or power density. Battery pack of may contain at least an electric conductor. In some cases, battery pack may include a plurality of electric conductors. Each electric conductor may provide electrical conductivity between one or more batteries or battery cells within battery pack. An “electric conductor,” as used in this disclosure, is an object or type of material that conducts a flow of charge or electric current. In a non-limiting example, an electric conductor may be continuous conductor. Exemplary battery cells in battery pack may include, lithium-ion battery cells, lithium-metal battery cells, air-metal battery cells, lead-acid battery cells, or the like. In some embodiment, battery pack may include a battery regulator. As described in this disclosure, a “battery regulator” is an electric device in a battery pack that performs battery regulation or redistribution. As used in this disclosure, “battery regulation” or “battery redistribution” refers to a process that keep voltage of each individual cell below its maximum value during operation, non-operation, or charging. In some embodiment, battery pack may include a battery balancer. As described herein, a “battery balancer” is an electric device in the battery pack that performs battery balancing. As used in this disclosure, “battery balancing” refers to a process that balances electric energy from one or more first battery cells (e.g., strong battery cells) to one or more second battery cells (e.g., weaker battery cells). Battery pack may be an inline package, wherein a plurality of battery cells is selected and stacked with solder in between them.

With continued reference to FIG. 1, processor 104 within apparatus 100 is configured to receive at least a biomarker 112 pertaining to the user from biological sensor 108. As used in this disclosure, “receive” from biological sensor 108 means accepting, collecting, or otherwise receiving input from biological sensor 108. In some cases, processor 104 may receive one or more biomarker 112 from the user. In a non-limiting example, at least a biomarker 112 may be manually input into processor 104 by a person. In another non-limiting example, at processor 104 may receive at least a biomarker 112 from biological sensor 108 in a signal form or any other data type described in this disclosure. In some embodiments, biological sensor 108 may be electrically connected to processor 104. In a non-limiting example, processor 104 may be electrically connected to biological sensor 108 through one or more wire. Wire may be continuous conductor described further in this disclosure. In other embodiments, without limitation, processor 104 may be communicatively connect to biological sensor 108. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, apparatus and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In a non-limiting example, processor 104 may be communicatively connected to control circuit 108 wirelessly.

With continued reference to FIG. 1, in some embodiments, processor 104 may be remote to biological sensor 108 and communicatively connected with biological sensor 108 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the processor 104 and/or biological sensor 108 connect directly, dynamically, and non-hierarchically to as many other computing devices as possible. A “network topology” as used in this disclosure is an arrangement of elements of a communication network. In a non-limiting example, a processor 104 may send instructions of any processing step described in this disclosure to a biological sensor 108 through a wireless wide area network, wherein the processor 104 and the biological sensor 108 may be connected to the wireless wide area network. Processor 104 may further receive, without limitation, any data, information, signals, and/or the like described in this disclosure from biological sensor 108 through wireless wide area network.

With continued reference to FIG. 1, as used in this disclosure, a “signal” is any intelligible representation of data, for example from one device to another. A signal may include an optical signal, a hydraulic signal, a pneumatic signal, a mechanical, signal, an electric signal, a digital signal, an analog signal and the like. In some cases, a signal may be used to communicate with a computing device, for example by way of one or more ports. In some cases, a signal may be transmitted and/or received by a computing device for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With continued reference to FIG. 1, in some cases, apparatus 100 may perform one or more signal processing steps on a signal. For instance, apparatus 100 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which varying continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables.

With continued reference to FIG. 1, processor 104 within apparatus 100 is further configured to determine a human performance measurement 136 pertaining to the user as a function of at least a biomarker 112. As used in this disclosure, a “human performance measurement” is a quantitative measurement relates to at least a person's ability to act. In a non-limiting example, human performance measurement 136 may include a measurement related to a person's ability to perform or complete a task 144. Task disclosed here will be described in further detail below. In some cases, human performance measurement may be gauged objectively. In some embodiments, human performance measurement 136 may include a FACTS parameter 140. As used in this disclosure, a “FACTS parameter” is a set of measurements related to foundation fields of human wellness. In some embodiments, without limitation, FACTS parameter may include a cognitive performance measurement, wherein the cognitive performance measurements measure cognitive abilities needed in acquisition of knowledge, manipulation of information, logic reasoning and the like thereof. In some embodiments, without limitation, FACTS parameter may include a workload measurement, wherein the workload measurement measures the amount of workload a user is capable of without overloading. In some embodiments, without limitation, FACTS parameter may include a fatigue measurement, wherein the fatigue measurement measures mental and/or physically tiredness of a user. In some embodiments, without limitation, FACTS parameter may include an anxiety measurement, wherein the anxiety measurement measures stressfulness of a user. In some embodiments, without limitation, FACTS parameter may include a confidence measurement, wherein the confidence measurement measures certainty of a user about an object, such as, without limitation, himself/herself, task 144, and the like thereof. In some embodiments, without limitation, FACTS parameter may include a trust measurement, wherein the trust measurement measures reliability of a user. In some embodiments, without limitation, FACTS parameter may include a sickness measurement, wherein the sickness measurement measures overall health of a user. In a non-limiting example, anxiety parameter may be derived using computing device from one or more heart rate measurements and/or breath frequency measured through biological sensor 108. In another non-limiting example, sickness measurement may be derived using computing device from one or more blood pressure measurements measured through biological sensor 108. In some embodiments, FACTS parameter may be determined as a function of one or more other FACTS parameters. In a non-limiting example, fatigue measurement may be determined as a function of workload measurement and/or sickness measurement.

With continued reference to FIG. 1, in some embodiments, determining human performance measurement 136 may include training a machine-learning process 128 using a biological training data 132, wherein the biological training data 132 may include a plurality of biomarkers such as, without limitation, behavioral indicia 116, oxygen delivery parameter 120, hematological parameter 124, and/or the like as input and correlated with a plurality of human performances measurements 136 such as, without limitation, FACTS parameter (i.e., cognitive performance measurement, workload measurement, fatigue measurement, anxiety measurement, confidence measurement, trust measurement, sickness measurement, and/or the like), success likelihood measurement 148, and/or acceptable human performance range as output and determining the human performance measurement 136 as a function of the trained Machine-learning process 128. In some cases, biological training data 132 may be obtained from a data store. In some cases, a data store may be a database. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. Additionally, or alternatively, in some embodiments, biological training data 132 may include manually labeled data. As a non-limiting example, biomarkers may be manually collected and labeled by the user and/or a medical professional. In some cases, biological training data may be compiled from historic information, for instance by a user. In some cases, historical information may include information captured from use of apparatus 100. In other cases, historical information may include a plurality of previous determinations made by machine-learning process 128 as feedback. In some embodiments, machine-learning process 128 may be used to determine a plurality of biomarkers; this may be performed using, without limitation, linear regression model, least squares regression, ridge regression, least absolute shrinkage and selection operator (LASSO) model, multi-task LASSO model, elastic net model, multi-task elastic net model, least angle regression (LAR), LARS LASSO model, orthogonal matching pursuit model, Bayesian regression, logistic regression, stochastic gradient descent model, perceptron model, passive aggressive algorithm. Robustness regression model, Huber regression model, or any other suitable model that may occur to person skilled in the art upon reviewing the entirety of this disclosure.

With continued reference to FIG. 1, processor 104 is further configured to classify a task 144 pertaining to the user as a function of human performance measurement 136. As used in this disclosure, a “task” is a piece of work to be done. In some cases, task 144 may include, without limitation, firefighting, resuscitation, investigating, combat, delivery, perform surgery, and the like thereof. In some cases, task 144 may include an acceptable human performance range, wherein the acceptable human performance range is a set of human performance measurements that are suitable for performing a given task. In a non-limiting example, task 144 may include an acceptable human performance range from a first human performance measurement to a second human performance measurement, wherein the second human performance measurement may be larger in magnitude comparing to the first human performance measurement, and wherein all human performance measurements in between first human performance measurement and second human performance measurement may be able to operate task 144. In a non-limiting example, classifying task 144 pertaining to the user may include training a task machine-learning process using task training data, wherein the task training data may include a plurality of human performance measurements as input and correlated with a plurality of tasks as output and classifying the human performance measurement 136 to task 144 as a function of the trained task machine-learning process. In some embodiments, separate machine-learning process may be a classifier, wherein the classifier may be used to classify a plurality of human performance measurements to one or more tasks 144. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a processes whereby a processor 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. In a non-limiting example, human performance measurement 136 may include a human performance vector x representing a FACTS parameter 140. [0.92, 0.95, 0.97, 0.90, 0.98, 0.95, 0.91], wherein the human performance vector x may include a plurality of measurements (i.e., cognitive performance measurement, workload measurement, fatigue measurement, anxiety measurement, confidence measurement, trust measurement, and sickness measurement) within the FACTS parameter 140 scale from 0 to 1. Task machine-learning process may take human performance vector x as input and output a task vector y [“Flight Control”, “Performing Surgery”, “Bomb disposal” ], wherein the task vector y include one or more suitable tasks 144 for such human performance measurement 136/human performance vector x.

With continued reference to FIG. 1, in some embodiments, human performance measurement 136 may include a success likelihood measurement 148. In some cases, success likelihood measurement may be estimated as a function of task 144 and human performance measurement 136 of the user. As used in this disclosure, a “success likelihood measurement” is an element representing a possibility of a user completing an assigned task as targeted. Assigned task may include, without limitation, task 144 described above. In some embodiments, success likelihood measurement may be on a scale with a numerical range. In a non-limiting example, success likelihood measurement may be in a scale from one to ten, wherein one may represent a failure and ten represent a success. In other embodiments, success likelihood measurement may be a probability. In a non-limiting example, success likelihood measurement of a user may be within 0% to 100%. In other cases, success likelihood measurement 148 may be predicted as a function of task, human performance measurements, and trained machine-learning process 128 as described above in this disclosure. In a non-limiting example, success likelihood measurement 148 may be estimated using trained machine-learning process 128 using task 144 and human performance measurement 136 pertaining to the user as input. Estimating success likelihood measurement 148 may include using any machine-learning algorithm described in this disclosure.

With continued reference to FIG. 1, in some embodiments, processor 104 is further configured to certify the user to perform task 144 as a function of the human performance measurement. In some embodiments, certifying the user to perform task 144 may include verifying human performance measurement 136 with acceptable human performance range of task 144. As used in this disclosure, “verifying” means comparing two objects. In a non-limiting example, a user may be allowed to perform task 144 if and only if the user's human performance measurement is within acceptable human performance range of task 144 as described above in this disclosure. In another non-limiting example, task 144 may include a human performance measurement requirement, wherein the human performance measurement requirement is a minimum human performance measurement for complete task 144 at a minimum level. Minimum level may vary based on task 144. In a non-limiting example, task such as performing a surgery may include a high human performance measurement 136. In another non-limiting example, task such as delivery may include a low human performance measurement 136. Additionally, or alternatively, certifying the user to perform task 144 may include verifying success likelihood measurement 148. In a non-limiting example, for a task with high risk, a success likelihood measurement over certain percentage such as, without limitation, 98% may be required for performing the task. Further Processor 104 may then authenticate the user for performing task 144 as a function of verification of human performance measurement 136. In a non-limiting example, a user may be unable to start on a task classified using machine-learning process if the user's human performance measurement is below a human performance measurement requirement of the task. In another non-limiting example, a user may be allowed to proceed to a task classified using machine-learning process if the user's human performance measurement is equal and/or above a human performance measurement requirement of the task.

Referring now to FIGS. 2A-C, an exemplary embodiment of a perspective view of a headset with biomarker measurement capabilities is illustrated in FIG. 2A. An exemplary embodiment of a front view of a headset with biomarker measurement capabilities is illustrated in FIG. 2B. An exemplary embodiment of a perspective view of a headset with biomarker measurement capabilities is illustrated in FIG. 2C.

Still referring to FIGS. 2A-C, one or more of biological sensor 108 may be attached to a housing 204. In an embodiment, attachment to housing may include mounting on an exterior surface of housing or seal. In some embodiments a biological sensor 108 may be incorporated within housing 204. Biological sensor 108 may additionally be electrically connected to another element within housing 204, or the like. Alternatively, or additionally, at least a biological sensor 108 may include a sensor that is not attached to housing 204 or is indirectly attached via wiring or the like. As a non-limiting example, at least a biological sensor 108 and/or one or more components thereof may be coupled to the substantially pliable seal 208. In an embodiment, at least a biological sensor 108 may be contacting exterior body surface; this may include direct contact with the exterior body surface, or indirect contact for instance through a portion of seal 208 or other components of apparatus 100. As a non-limiting example of placement of at least a biological sensor 108, and as illustrated for exemplary purposes in FIGS. 2, at least a biological sensor 108 may include a sensor mounted on an edge of an earcup, and so positioned that placement of earcup over user's ear places sensor in contact with user's skin just behind the ear at a local skeletal eminence. Similarly, where housing 204 includes a mask as described above, a sensor of at least a biological sensor 108 may be disposed within mask at a location that, when mask is worn, places sensor against a forehead of user.

Still referring to FIG. 2A, Housing 204 may include a rigid outer shell 204. Rigid outer shell 204 may, for instance, protect internal elements of headset 200 from damage, and maintain them in a correct position on a user's body. Housing 204 and/or rigid outer shell 204 may be inserted on a head of the user, in particular the housing 204 may cover the ears of the user. As a non-limiting example, exterior body surface may be a surface, such as a surface of the head, face, or neck of user, which is wholly or partially covered by helmet, as described for example in further detail below. As a further non-limiting example, housing 204 may be formed to have a similar or identical shape to a standard-issue “ear cup” incorporated in an aviation helmet, so that housing 204 can replace ear cup after ear cup has been removed. Headset 200 may be the same or substantially the same as apparatus 100.

Still referring to FIG. 2A, Seal 208 may be substantially pliable; seal 208 may be constructed of elastomeric, elastic, or flexible materials including without limitation flexible, elastomeric, or elastic rubber, plastic, silicone including medical grade silicone, gel, and the like. Substantially pliable seal 208 may include any combination of materials demonstrating flexible, elastomeric, or elastic properties, including without limitation foams covered with flexible membranes or sheets of polymer, leather, or textile material. As a non-limiting example, substantially pliable seal 208 may include any suitable pliable material for placement over a user's ear, including without limitation any pliable material or combination of materials suitable for use on headphones, headsets, earbuds, or the like. In an embodiment, substantially pliable seal 208 advantageously aids in maintaining housing 204 and/or other components of headset 200 against exterior body surface; for instance, where exterior body surface has elastomeric properties and may be expected to flex, stretch, or otherwise alter its shape or position to during operation, substantially pliable seal 208 may also stretch, flex, or otherwise alter its shape similarly under similar conditions, which may have the effect of maintaining seal 208 and/or one or more components of apparatus 100 as described in greater detail below. Seal 208 may be attached to housing 204 by any suitable means, including without limitation adhesion, fastening by stitching, stapling, or other penetrative means, snapping together or otherwise engaging interlocking parts, or the like. Seal 208 may be removably attached to housing 204, where removable attachment signifies attachment according to a process that permits repeated attachment and detachment without noticeable damage to housing 204 and/or seal 208, and without noticeable impairment of an ability to reattach again by the same process. As a non-limiting example, substantially pliable seal 208 may be placed on an ear cup of the housing 204.

With continued reference to FIGS. 2B, housing 204 may be incorporated into a headset. A headset may include, without limitation, an aviation headset, such as headsets as manufactured by the David Clark company of Worcester Massachusetts, or similar apparatuses. A headset may also be used commercially for recreational use or fitness use. In some embodiments, housing 204 is headset; that is, apparatus 100 may be manufactured by incorporating one or more components into the headset, using the headset as a housing 204. As a further non-limiting example, housing 204 may include a mask; a mask as used herein may include any device or element of clothing that is worn on a face of user during operation, occluding at least a part of the face. Masks may include, without limitation, safety googles, gas masks, dust masks, self-contained breathing apparatuses (SCBA), self-contained underwater breathing apparatuses (SCUBA), and/or other devices worn on and at least partially occluding the face for safety, functional, or aesthetic purposes. Apparatus 100 may include a mask. Headset 200 may be manufactured by incorporating one or more elements or components of a mask in or on headset 200.

With continued reference to FIGS. 2B, a plurality of Housings 204 may attach to an element of headgear 212. As used in the current disclosure, a “headgear” is any element worn on and partially occluding a head or cranium of user. In an embodiment, a headgear 212 may attach two housings 204 in a manner which they may be worn around the head. Headgear 212 may wholly or partially occlude user's face and thus also include a mask; headgear 212 may include, for instance, a fully enclosed diving helmet, space helmet, or helmet incorporated in a space suit, or the like. Headgear 212 may include a headband, such as without limitation a headband of a headset. As used in the current disclosure, a “headband” is a band in a horseshoe shape configured to be worn over the top of the head of the user. Additionally, the headband may be connecting piece that runs from a first housing to a second housing. The headband may hold them together so that you can comfortably and securely wear the headset 200 on your head. In an embodiment, the housing 204 may be electrically connected by running a wire through the headband. Headgear 212 may include a hat, a helmet, a construction “hardhat,” a bicycle helmet, or the like.

With continued reference to FIG. 2C, housing 204 may house a plurality of hardware associated with apparatus 100 including processor 104, biological sensor 108, and the like may be located within the housing. Housing 204 may be configured mounted to an exterior body surface of a user; exterior body surface may include, without limitation, skin, hair, an interior surface of an orifice such as the mouth, nose, or ears, or the like. Exterior body surface and/or locus may include an exterior body surface of user's head, face, or neck. In a non-limiting example, biological sensor may include an environmental temperature sensor (i.e., cutaneous sensor), wherein the environmental temperature sensor may be positioned to within earcup to measure temperature of ambient air proximal skin surface, thereby providing a relative temperature measurement for skin temperature.

With continued reference to FIG. 2C, in an embodiment, at least a cutaneous biological sensor 108 may contact a locus on the exterior body surface where substantially no muscle is located between the exterior body surface and an underlying bone structure, meaning muscle is not located between the exterior body surface and an underlying bone structure and/or any muscle tissue located there is unnoticeable to a user as a muscle and/or incapable of appreciably flexing or changing its width in response to neural signals; such a locus may include, as a non-limiting example, locations on the upper cranium, forehead, nose, behind the ear, at the end of an elbow, on a kneecap, at the coccyx, or the like. Location at a locus where muscle is not located between exterior body surface and underlying bone structure may decrease reading interference and/or inaccuracies created by movement and flexing of muscular tissue. At least a biological sensor 108 may contact a locus having little or no hair on top of skin. At least a biological sensor 108 may contact a locus near to a blood vessel, such as a locus where a large artery such as the carotid artery or a branch thereof, or a large vein such as the jugular vein, runs near to skin or bone at the location; in an embodiment, such a position may permit at least a biological sensor 108 to detect circulatory parameters as described above.

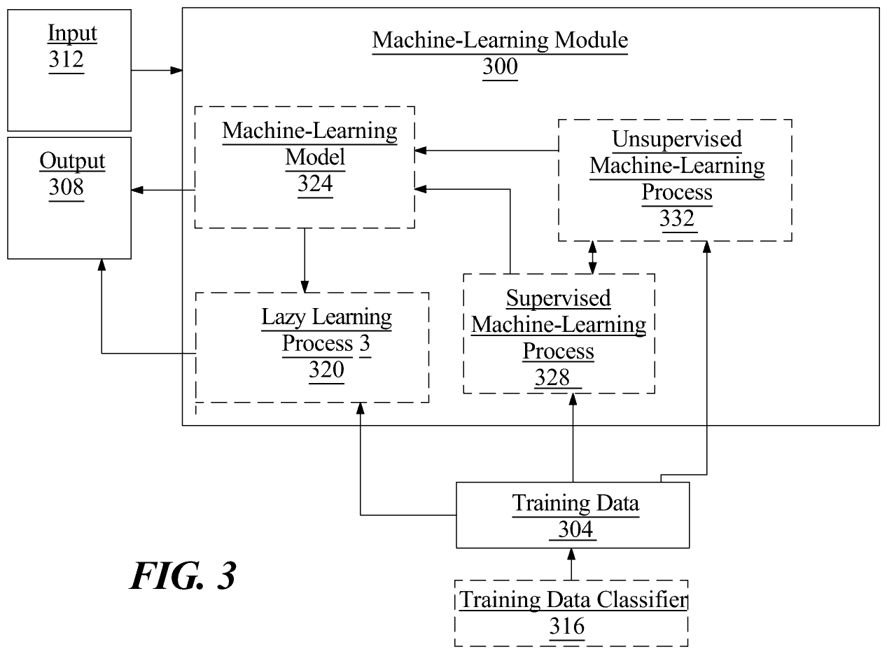

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example inputs may include a plurality of electric potential and time and outputs may include a plurality of current values.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 300 may generate a classifier using a classification algorithm, defined as a process whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to biomarker 112 such as without limitation, behavioral indicia 116, oxygen delivery parameter 120, hematological parameter 124 and the like, human performance measurement 136, FACTS parameter 140, and Task 144.

With continued reference to FIG. 3, Processor 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, processor 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 4]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where a; is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 3, machine-learning processes as described in this disclosure may be used to generate machine-learning models 324. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described above in this disclosure as inputs, outputs as described above in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

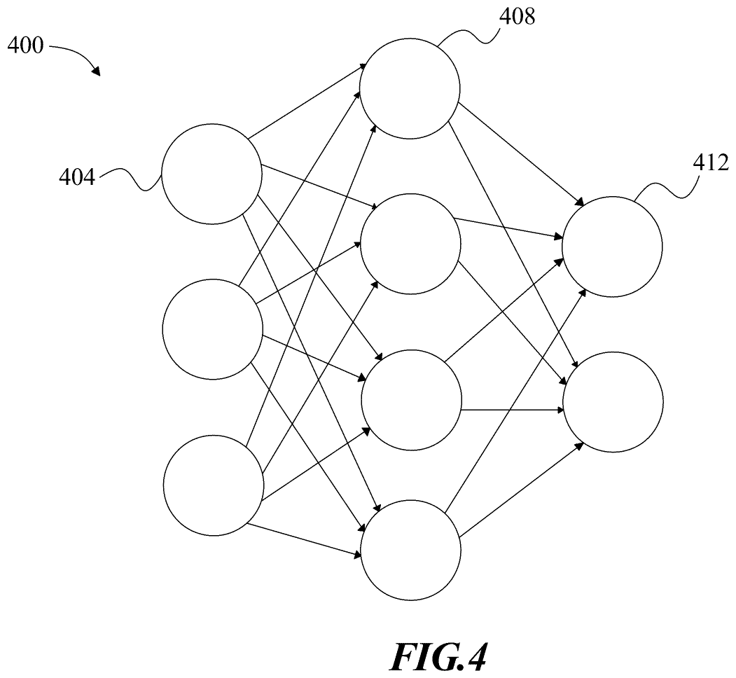

Referring now to FIG. 4, an exemplary embodiment of neural network 500 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

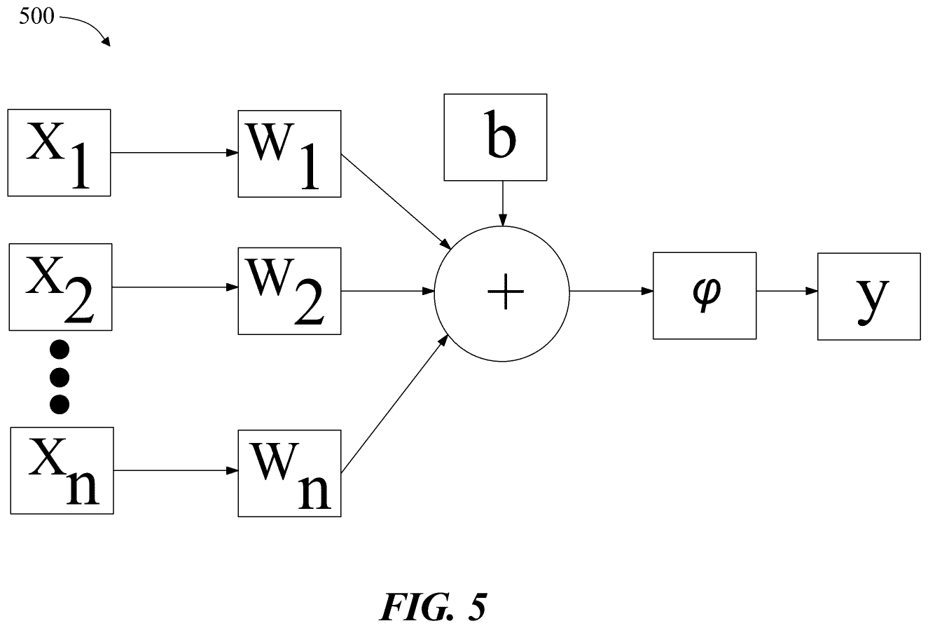

Referring now to FIG. 5, an exemplary embodiment of a node of a neural network is illustrated. A node may include, without limitation a plurality of inputs x; that may receive numerical values from inputs to a neural network containing the node and/or from other nodes. Node may perform a weighted sum of inputs using weights w, that are multiplied by respective inputs xi. Additionally, or alternatively, a bias b may be added to the weighted sum of the inputs such that an offset is added to each unit in the neural network layer that is independent of the input to the layer. The weighted sum may then be input into a function p, which may generate one or more outputs y. Weight w, applied to an input x; may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wi may be determined by training a neural network using training data, which may be performed using any suitable process as described above.

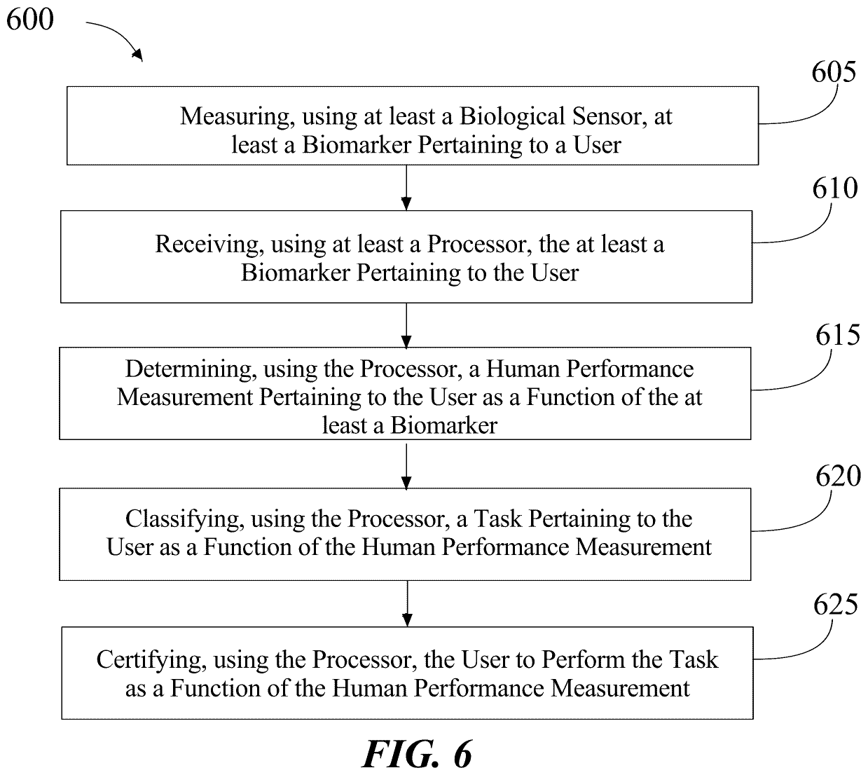

Referring now to FIG. 6, an exemplary embodiment of a method 600 for calculating human performance is illustrated. Method 600 includes a step 605 of measuring, using at least a biological sensor, at least a biomarker pertaining to a user, without limitation, as described above in reference to FIGS. 1-5. Biological sensor may be any sensor described in this disclosure. In some embodiments, biomarker may include a behavioral indicia. In some embodiments, biomarker may further include an oxygen delivery parameter. In some embodiments, biomarker may further include at least a hematological parameter pertaining to the user. This may be implemented, without limitation, as described above in reference to FIGS. 1-5.

With continued reference to FIG. 6, method 600 further include a step 610 of receiving, using at least a processor, the at least a biomarker pertaining to the user from at least a biological sensor, without limitation, as described above in reference to FIGS. 1-5. Processor may be any computing device described in this disclosure. biological sensor is communicatively connected to at least a processor. This may be implemented, without limitation, as described in reference to FIGS. 1-5.

With continued reference to FIG. 6, method 600 further include a step 615 of determining, using the processor, a human performance measurement pertaining to the user as a function of the at least a biomarker. In some embodiments, human performance measurement may include a FACTS parameter. In some embodiments, human performance measurement may further include a success likelihood measurement pertaining to the user. This may be implemented, without limitation, as described above in reference to FIGS. 1-5. In some embodiments, step 615 of determining the human performance measurement may include training a machine-learning process using a biological training data, wherein the biological training data may include a plurality of biomarkers as input and correlated with a plurality of human performances as output and determining the human performance measurement as a function the trained machine-learning process. This may be implemented, without limitation, as described above in reference to FIGS. 1-5.

With continued reference to FIG. 6, method 600 further include a step 620 of classifying, using the processor, a task pertaining to the user as a function of human performance measurement, without limitation, as described above in reference to FIGS. 1-5. In some embodiments, task may include an acceptable human performance range. In some embodiments, step 620 of classifying task pertaining to the user may include training a task machine-learning process using a task training data, wherein the task training data may include a plurality of human performance measurements as input and correlated with a plurality of tasks as output and classifying task as a function of the trained task machine-learning process. This may be implemented, without limitation, as described above in reference to FIGS. 1-5.

With continued reference to FIG. 6, method 600 further include a step 625 of certifying, using the processor, the user to perform task as a function of the human performance measurement, without limitation, as described above in reference to FIGS. 1-5. In some embodiments, step 625 of certifying the user may include verifying human performance measurement pertaining to the user with acceptable human performance range and authenticate the user for performing task as a function of the verification of the human performance measurement. In other embodiments, step 625 of certifying the user may include verifying success likelihood measurement pertaining to the user. This may be implemented, without limitation, as described above in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

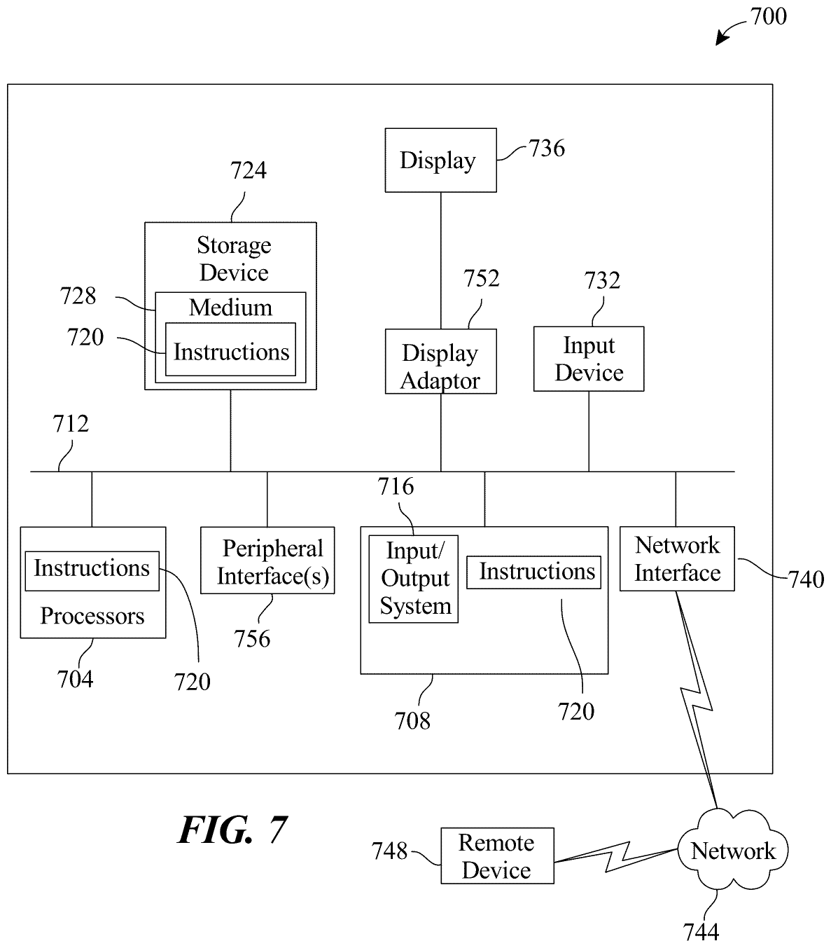

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, apparatus, system and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for calculating human performance, wherein the apparatus comprises:
at least a biological sensor configured to:
measure at least a biomarker pertaining to a user;
at least a processor communicatively connected to the at least a biological sensor; and
a memory communicatively connected to the at least a processor, wherein the memory contains instructions configuring the at least a processor to:
receive the at least a biomarker pertaining to the user from the at least a biological sensor; and
determine a human performance measurement pertaining to the user as a function of the at least a biomarker, wherein determining the human performance measurement further comprises:
train a machine-learning process iteratively using a biological training data, wherein the biological training data comprises the biomarker as inputs and correlated with a plurality of human performance measurements as outputs, wherein the biological training data further comprises a plurality of previously determined correlations made by the machine-learning process as feedback; and
determine the human performance measurement as a function of the trained machine-learning process;
classify a task pertaining to the user as a function of the human performance measurement; and
certify the user to perform the task as a function of the human performance measurement.

2. The apparatus of claim 1, wherein the biomarker comprises behavioral indicia.

3. The apparatus of claim 1, wherein the biomarker comprises an oxygen delivery parameter.

4. The apparatus of claim 1, wherein the biomarker comprises a plurality of hematological parameters.

5. The apparatus of claim 1, wherein the human performance measurement comprises a FACTS parameter.

6. The apparatus of claim 1, wherein the human performance measurement comprises a success likelihood measurement.

7. The apparatus of claim 1, wherein the task comprises an acceptable human performance range, wherein the acceptable human performance range comprises a set of human performance measurements that are suitable for performing the task.

8. (canceled)

9. The apparatus of claim 1, wherein classifying the task pertaining to the user comprises:
training a task machine-learning process using a task training data, wherein the task training data comprises the plurality of human performance measurements as input and correlated with task as output; and
classifying the task pertaining to the user as a function of the trained task machine-learning process.

10. The apparatus of claim 7, wherein certifying the user comprises:
verify the human performance measurement pertaining to the user with the acceptable human performance range; and
authenticate the user for performing the task as a function of the verification of the human performance measurement.

11. A method for calculating human performance, wherein the method comprises:
measuring, using at least a biological sensor, at least a biomarker pertaining to a user;
receiving, using at least a processor, the at least a biomarker pertaining to the user from the at least a biological sensor;
determining, using the at least a processor, a human performance measurement pertaining to the user as a function of the at least a biomarker;
classifying, using the at least a processor, a task pertaining to the user as a function of the human performance measurement; and
certifying, using the at least a processor, the user to perform the task as a function of the human performance measurement.

12. The method of claim 11, wherein the biomarker comprises behavioral indicia.

13. The method of claim 11, wherein the biomarker comprises an oxygen delivery parameter.

14. The method of claim 11, wherein the biomarker comprises at least a hematological parameter.

15. The method of claim 11, wherein the human performance measurement comprises a FACTS parameter.

16. The method of claim 11, wherein the human performance measurement comprises a success likelihood measurement pertain to the user.

17. The method of claim 11, wherein the task comprises an acceptable human performance range.

18. The method of claim 11, wherein determining the human performance measurement comprises:
training a machine-learning process using a biological training data, wherein the biological training data comprises a plurality of biomarkers as input and correlated with a plurality of human performances as output; and
determining the human performance measurement as a function the trained machine-learning process.

19. The method of claim 11, wherein classifying the task pertaining to the user comprises:
training a task machine-learning process using a task training data, wherein the task training data comprises a plurality of human performance measurements as input and correlated with a plurality of tasks as output; and
classifying the task pertaining to the user as a function of the trained task machine-learning process.

20. The method of claim 17, wherein certifying the user comprises:
verifying the human performance measurement pertaining to the user with the acceptable human performance range; and
authenticating the user for performing the task as a function of the verification of the human performance measurement.
